(19) United States
(12) Patent Application Publication
(10) Pub. No.: US 2024/0159703 A1
(43) Pub. Date: May 16, 2024

(54) CAPILLARY ELECTROPHORESIS METHODS FOR QUANTIFYING VIRAL SPECIES

(71) Applicant: DH Technologies Development Pte. Ltd., Singapore (SG)

(72) Inventors: Tingting Li, Yorba Linda, CA (US); Handy Yowanto, Walnut, CA (US); Andras Guttman, San Diego, CA (US)

(21) Appl. No.: 18/548,708

(22) PCT Filed: Feb. 28, 2022
(86) PCT No.: PCT/IB2022/051754

§ 371 Date: Sep. 1, 2023

Related U.S. Application Data
(63) Continuation-in-part of application No. 63/155,891, filed on Mar. 3, 2021.

(51) Int. Cl. G01N 27/447; G01N 33/569

(52) U.S. Cl. CPC G01N 27/44726; G01N 27/44791; G01N 33/56983; G01N 2333/155; G01N 2458/00

(57) ABSTRACT

Methods and kits for quantifying a viral species are presently claimed and described. The method includes the steps of preparing at least one labeled viral protein component by incubating a detectable dye with a virial species in the presence of sodium dodecyl sulfate (SDS); loading the labeled viral protein component onto a capillary electrophoresis (CE) capillary, wherein the CE capillary is filled with a buffer comprising a polymer matrix; applying a separation voltage to the CE capillary; detecting at least one labeled viral protein component with a detector, thereby producing a corresponding set of values; and quantifying a protein of interest in the viral protein component using the corresponding set of values.

### RELATED APPLICATIONS

The present patent application claims the priority benefit of U.S. Provisional Patent Application Ser. No. 63/155,891, filed Mar. 3, 2021, the content of which is hereby incorporated by reference in its entirety into this disclosure.

### BACKGROUND

The various challenges during viral species production include virus stability, the virus' toxicity to the host cell lines, and limited ability to properly characterize the active virus product. As a result, monitoring product quality is critical. Titer is one of the critical quality attributes (CQA) for viral species production from lab scale to GMP manufacturing. Quantitation (titer determination) of a viral species to ensure efficient expression is usually based on the quantification of a protein of interest. An enzyme-linked immunosorbent assay (ELISA) is the traditional method for viral species titer determination. However, this method measures both virus-associated protein(s) of interest and possible false positives with similar epitopes, resulting in an overestimation of the titer. This calls for an orthogonal method to be used in combination with the regularly used screening ELISA test, such as a separation-based technique. The latter can be used to recognize false positives and provide more accurate titer and concomitantly utilized for profile-based purity assessment. Guzman, N. A. and D. E. Guzman, A Two-Dimensional Affinity Capture and Separation Mini-Platform for the Isolation, Enrichment, and Quantification of Biomarkers and Its Potential Use for Liquid Biopsy. Biomedicines, 2020. 8(8).

### SUMMARY

The inventors have recognized the need for high precision viral species titer determination and its protein profiling based purity assessment. The claimed and described approach offers an automated analysis of the viral species proteome with high resolution, excellent quantitation capability, and great reproducibility. An easily applicable two-step sample preparation and fluorescent labeling protocol is also described.

One aspect of the disclosure relates to a method for quantifying a viral species, the method comprising the steps of preparing at least one labeled viral protein component by incubating a detectable dye with a virial species in the presence of sodium dodecyl sulfate (SDS); loading the labeled viral protein component onto a capillary electrophoresis (CE) capillary, wherein the CE capillary is filled with a buffer comprising a polymer matrix; applying a separation voltage to the CE capillary; detecting at least one labeled viral protein component with a detector, thereby producing a corresponding set of values, and quantifying a protein of interest in the viral protein component using the corresponding set of values.

In one aspect, the viral species is a retrovirus. In another aspect, the viral species is a lentivirus.

In one aspect, the protein component is a structural protein, non-structural protein, or residual protein. In one aspect, the structural protein is a nucleocapsid protein, capsid protein, matrix protein, or envelope protein. In another aspect, the structural protein component is a p24 capsid protein.

In one aspect, the viral species is incubated at a temperature between about 40° C. to about 90° C., alternatively at a temperature between about 45° C. to about 85° C., alternatively at a temperature between about 50° C. to about 80° C., alternatively at a temperature between about 55° C. to about 78° C., alternatively at a temperature between about 60° C. to about 77° C., alternatively at a temperature between about 65° C. to about 75° C., alternatively at a temperature between about 68° C. to about 74° C., alternatively at a temperature between about 69° C. to about 73° C., alternatively at a temperature of about 70° C.

In another aspect, the viral species is incubated for at least 2 minutes, alternatively at least 5 minutes, alternatively at least 10 minutes, alternatively at least 15 minutes, alternatively at least 20 minutes, alternatively at least 25 minutes, alternatively at least 30 minutes, alternatively at least 35 minutes, alternatively at least 40 minutes, alternatively at least 45 minutes, alternatively at least 50 minutes, alternatively at least 55 minutes, alternatively at least 60 minutes.

In one aspect, the detectable dye is a fluorescent dye. In another aspect, the detectable dye has an absorption wavelength and an emission wavelength of between about 300 nm and about 750 nm. In a further aspect, the detectable dye is a pyrylium-based dye. In yet another aspect, the detectable dye is an amine-reactive pyrylium dye.

In one aspect, the polymer matrix is selected from the group consisting of crosslinked polymer, linear polymers, slightly branched polymers, linear polyacrylamide, polyethylene oxide, polyethylene glycol, dextran, and pullulan.

In one aspect, the detector is a fluorescence detector, preferably a laser induced fluorescence (LIF) detector.

In one aspect, the limit of quantification is in a low ng/mL range. In a further aspect, the limit of quantification is in a sub ng/mL range.

In one aspect, at least two labeled viral protein components are prepared, alternatively at least three labeled viral protein components are prepared, alternatively at least four labeled viral protein components are prepared, alternatively at least five labeled viral protein components are prepared, alternatively at least six labeled viral protein components are prepared, alternatively at least seven labeled viral protein components are prepared, alternatively at least eight labeled viral protein components are prepared, alternatively at least nine labeled viral protein components are prepared, alternatively at least ten labeled viral protein components are prepared.

One aspect of the disclosure relates to a kit for quantifying a viral species, wherein the kit comprises a CE capillary, a buffer comprising a polymer matrix; at least one sample buffer, wherein the sample buffer comprises sodium dodecyl sulfate (SDS); at least one internal standard; at least one wash solution; and instructions for use. In another aspect, the kit comprises a detectable dye. In a further aspect, the CE capillary is pre-filled with the buffer comprising a polymer matrix.

Other aspects and features of the present disclosure will become apparent to those ordinarily skilled in the art upon review of the following description of specific embodiments of the disclosure in conjunction with the accompanying figures.

### BRIEF DESCRIPTION OF THE FIGURES

Embodiments of the present disclosure will now be described, by way of example only, with reference to the attached Figures.

FIG. 1 illustrates a lentivirus structure. Titer determination is accomplished by quantifying the p24 capsid protein.

FIG. 2 depicts a sodium dodecyl sulfate capillary gel electrophoresis of pyrylium dye labeled lentivirus proteome. Conditions: EZ-CE cartridge with 30 cm total length (20 cm to the detection point, 50 μm ID) fused silica capillary filled with a buffer comprising a polymer matrix, E=500V/cm, temperature: 25° C., Injection parameters: water pre-injection for 0.4 min at 20 psi followed by sample injection for 1 min at 5 kV.

FIGS. 3A-3D depict the p24 determination of sodium dodecyl sulfate-capillary gel electrophoresis (SDS-CGE) analysis of the pyrylium dye labeled lentivirus proteome. The circled peak is the p24 protein for titer determination. The increasing amounts of spiked p24 are shown at the corresponding traces. The lentivirus concentration of FIGS. 3A and 3B is 1.0×10⁹ TU/mL and the lentivirus concentration of FIGS. 3C and 3D is 1.5×10⁸ TU/mL. Separation conditions were the same as in FIG. 2.

FIGS. 4A and 4B depict the linearity and limit of quantification based on the SDS-CGE analysis of the p24 protein.

FIGS. 5A and 5B depict the repeatability and reproducibility of the lentivirus titer determination detection based on the SDS-CGE analysis of the p24 protein.

FIGS. 6A and 6B depict repeatability and reproducibility the lentivirus titer determination detection of six different sample preparation based on the SDS-CGE analysis of the p24 protein.

FIG. 7A depicts the standard curve of a labeled viral protein component quantified on three separate days. FIG. 7B depicts a superimposed p24 standard at different concentrations. FIG. 7C depicts the average of the standard curves prepared and analyzed across three different days with the corresponding error bars. Separation conditions were the same as in FIG. 2.

FIG. 8 shows initial conditions in accordance with one aspect of the disclosure.

FIG. 9 shows LIF detector initial conditions in accordance with one aspect of the disclosure.

FIG. 10 shows a time program for conditioning in accordance with one aspect of the disclosure.

FIG. 11 shows a time program for separation in accordance with one aspect of the disclosure.

### DETAILED DESCRIPTION

It is to be understood that this disclosure is not limited to the particular methodology, protocols, and reagents described herein and as such may vary. It is also to be understood that the terminology used herein is for the purpose of describing particular embodiments only and is not intended to limit the scope of the present disclosure or the appended claims.

As used herein and in the appended claims, the singular forms “a,” “an,” and “the” include plural reference unless the context clearly indicates otherwise. The singular form “a”, “an” and “the” include plural referents unless the context clearly dictates otherwise. These articles refer to one or to more than one (i.e., to at least one). The term “and/or” means any one or more of the items in the list joined by “and/or”. As an example, “x and/or y” means any element of the three-element set {(x), (y), (x, y)}. In other words, “x and/or y” means “one or both of x and y”. As another example, “x, y, and/or z” means any element of the seven-element set {(x), (y), (z), (x, y), (x, z), (y, z), (x, y, z)}. In other words, “x, y and/or z” means “one or more of x, y and z”.

The term “about” is used in connection with a numerical value throughout the specification and the claims denote an interval of accuracy, familiar and acceptable to a person skilled in the art. In general, such an interval of accuracy is +/−10%.

The term “exemplary” means serving as a non-limiting example, instance, or illustration. As utilized herein, the terms “e.g.,” and “for example” set off lists of one or more non-limiting aspects, examples, instances, or illustrations.

The term “low ng/mL range” includes amounts from about 50 ng/mL-about 5000 ng/mL. The term “sub ng/mL range” includes amounts from about 0.01 ng/mL-about 49.99 ng/mL.

Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

Unless defined otherwise, all technical and scientific terms used herein have the same meaning as commonly understood to one of ordinary skill in the art to which this disclosure belongs.

Embodiments of this disclosure include methods for a high-performance sodium dodecyl sulfate capillary gel electrophoresis based assay, using a capillary electrophoresis platform in conjunction with sodium dodecyl sulfate-molecular weight protein analysis. Embodiments of this disclosure can be used for highly precise viral titer determination and profiling. The disclosed methods allow for the separation of proteins in the size range of about 10 kDa to about 225 kDa with high resolution, excellent quantitation capability, and great reproducibility. In addition to the separation methods, the method further comprises steps for denaturing and labeling a viral species for quantitative determination of a protein of interest using a detectable dye.

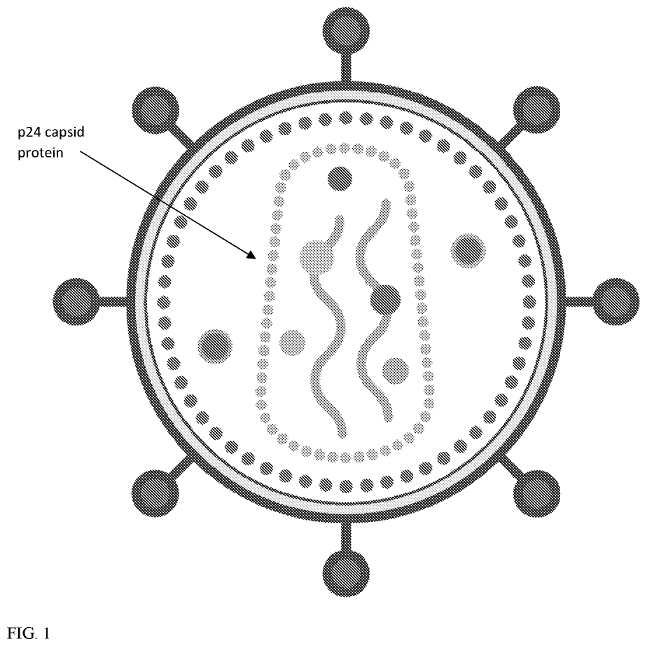

Non-limiting examples of viral species quantified using an embodiment of the disclosure include retrovirus or lentivirus. Lentivirus (depicted in FIG. 1) can integrate large DNA molecules into host cells, thus representing one of the most efficient gene delivery vehicles for transduction. Cockrell, A. S. and T. Kafri, Gene delivery by lentivirus vectors. Mol Biotechnol, 2007. 36(3): p. 184-204. For this particular application, lentivirus has several advantages, including its ability to infect non-dividing and dividing cells. For clinical therapeutic applications, the stable and long term transgene expression of these retroviruses represent a great advantage. Anguela, X. M. and K. A. High, Entering the Modern Era of Gene Therapy. Annu Rev Med, 2019. 70: p. 273-288. The proteome of the lentivirus comprises five important structural and several non-structural proteins. The vital structural proteins are gp120 surface envelope protein (120 kDa), gp41 transmembrane envelope protein (41 kDa), p24 capsid protein (24 kDa), p17 matrix protein (17 kDa), and the p7/P9 nucleocapsid protein (7-11 kDa). The two envelope proteins of gp120 and gp41 are highly glycosylated. This post-translational modification (PTM) apparently conceals important antigenic sites, thus helps the virus to avoid recognition by the immune system of the host. The p24 protein is the building block of the lentivirus capsid, and approximately 2000 of them assemble the full capsid.

One embodiment of the disclosure is a method for quantifying a viral species, the method comprising the steps of preparing a labeled viral protein component by incubating a detectable dye with a virial species in the presence of sodium dodecyl sulfate (SDS); loading the labeled viral protein component onto a capillary electrophoresis (CE) capillary, wherein the CE capillary is filled with a buffer comprising a polymer matrix; applying a separation voltage to the CE capillary; detecting at least one labeled viral protein component with a detector, thereby producing a corresponding set of values; and quantifying a protein of interest in the viral protein component using the corresponding set of values.

In an embodiment where the viral species is a lentivirus, the protein component detected is a structural protein, such as a nucleocapsid protein, capsid protein, matrix protein, or envelope protein. In an embodiment, the capsid protein is a p24 capsid protein. In some embodiments, the protein component detected is a non-structural protein or a residual protein.

In an embodiment, the method comprises an efficient two-step sample preparation protocol, including the denaturation and covalent labeling of the protein of interest with a detectable dye. The detectable dye may be a fluorescent dye, and includes dyes that independently have an absorption wavelength and emission wavelength of between about 480 nm and about 740 nm, alternatively about 490 nm, alternatively about 500 nm, alternatively about 510 nm, alternatively about 520 nm, alternatively about 530 nm, alternatively about 540 nm, alternatively about 550 nm, alternatively about 560 nm, alternatively about 570 nm, alternatively about 580 nm, alternatively about 590 nm, alternatively about 600 nm, alternatively about 610 nm, alternatively about 620 nm, alternatively about 630 nm, alternatively about 640 nm, alternatively about 650 nm, alternatively about 660 nm, alternatively about 670 nm, alternatively about 680 nm, alternatively about 690 nm, alternatively about 700 nm, alternatively about 710 nm, alternatively about 720 nm, alternatively about 730 nm Non-limiting examples of the detectable dye include a pyrylium-based dye and an amine-reactive pyrylium dye. Pyrylium dyes fluoresce when the dye reacts with proteins. Amine-reactive pyrylium dye fluoresce when the dye readily reacts with the primary amines of the polypeptide backbone of proteins and features very low (<1%) fluorescence in its unconjugated format, thus, decreasing baseline noise and increasing detection limit. The use of pyrylium-based dyes also retains a protein's natural charge state.

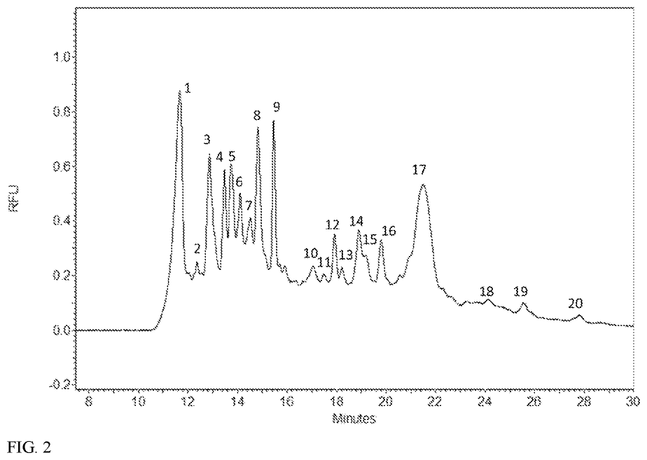

In an exemplary embodiment, a lentivirus proteome is labeled with a pyrylium dye and was then subject to quantitative sodium dodecyl sulfate capillary gel electrophoresis analysis. The resulting electropherogram is shown in FIG. 2 and features over 20 peaks. Since most of the lentivirus proteins possess various post-translational modifications (PTMs), such as glycosylation, standard curve based molecular mass assessment using the sizing ladder is not considered adequate.

The detectable dye is incubated with the viral species for a time sufficient to allow for the formation of a labeled viral protein component. In various embodiments, the viral species is incubated for at least 2 minutes, alternatively at least 5 minutes, alternatively at least 10 minutes, alternatively at least 15 minutes, alternatively at least 20 minutes, alternatively at least 25 minutes, alternatively at least 30 minutes, alternatively at least 35 minutes, alternatively at least 40 minutes, alternatively at least 45 minutes, alternatively at least 50 minutes, alternatively at least 55 minutes, alternatively at least 60 minutes.

The detectable dye is also incubated with the viral species at a temperature sufficient to allow for the formation of a labeled viral protein component. In various embodiments, the viral species is incubated at a temperature between about 40° C. to about 90° C., alternatively at a temperature between about 45° C. to about 85° C., alternatively at a temperature between about 50° C. to about 80° C., alternatively at a temperature between about 55° C. to about 78° C., alternatively at a temperature between about 60° C. to about 77° C., alternatively at a temperature between about 65° C. to about 75° C., alternatively at a temperature between about 68° C. to about 74° C., alternatively at a temperature between about 69° C. to about 73° C., alternatively at a temperature of about 70° C.

In an embodiment, the labeled viral protein component is loaded onto a capillary electrophoresis (CE) capillary. The CE capillary may be filled with a buffer comprising a polymer matrix or gel buffer prior to applying a separation voltage and/or loading the labeled viral protein component. In some embodiments, the buffer comprising a polymer matrix or gel buffer is placed into a buffer vial. These buffer vials may be placed into buffer trays. In some embodiments, the buffer comprising a polymer matrix or gel buffer may comprise additional components to facilitate the separation of the protein and/or protein components of interest. Non-limiting examples of a suitable polymer matrix include crosslinked polymer, linear polymers, slightly branched polymers, linear polyacrylamide, polyethylene oxide, polyethylene glycol, dextran, and pullulan.

In an embodiment, a separation voltage is applied to the CE capillary, and the labeled viral protein components are moved towards a detector. The detector can be a UV detector or a fluorescence detector, such as a laser induced fluorescence (LIF) detector, a lamp-based fluorescence detector, or a native fluorescence detector. The desired quantitation sensitivity will determine the type of detector used. LIF detection offers the benefit of about a 100-fold increase in sensitivity, yet it also requires additional sample manipulation.

Once the labeled viral protein components are detected, a corresponding set of values is generated. In an embodiment, these values may be plotted on an electropherogram. Once plotted on an electropherogram, at least one viral protein component from the labeled protein sample may be identified (e.g., qualitative assay). Qualitative analysis may also be used to estimate the molecular weights of the protein components. In various embodiments, the limits of detection are in the low ng/mL range, alternatively in the sub ng/mL range.

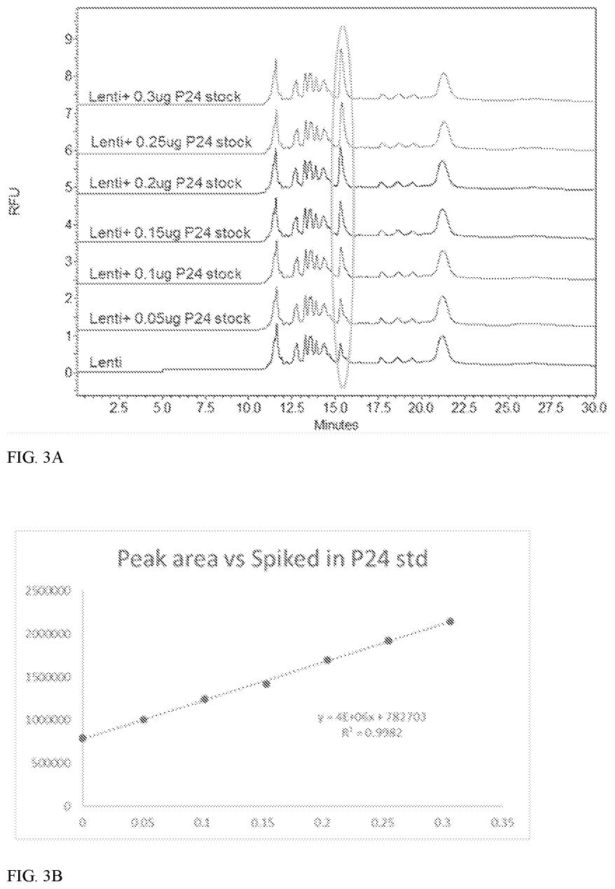

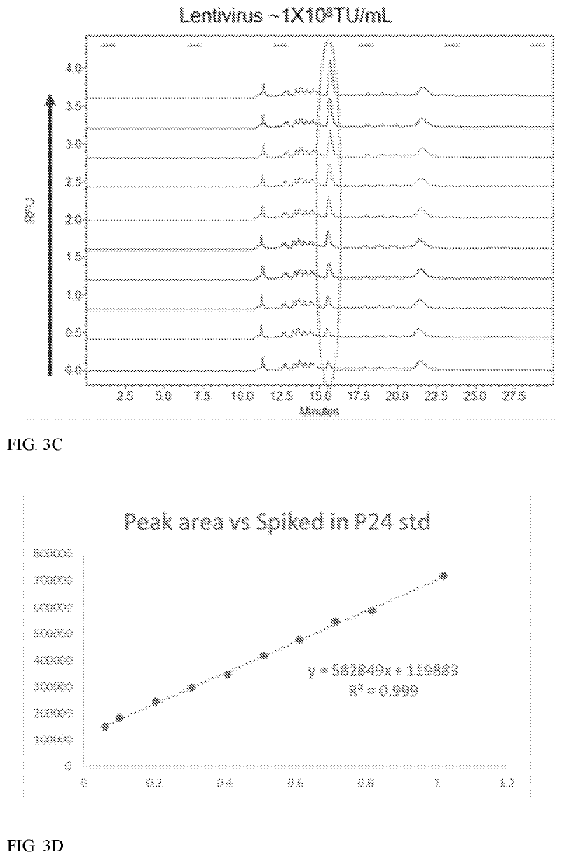

In an embodiment, the corresponding set of values is used to quantitate the viral protein components and/or used for viral titer determination. In an exemplary embodiment, the peak corresponding to the p24 protein for titer determination was determined by spiking the sample with a commercially available standard. FIGS. 3A and 3C compare the original separation trace with six spiked SDS-CGE electropherograms reflecting the effect of the increasing amount of the p24 protein from 0.05 μg to 0.3 μg (FIG. 3A) or from 0.003 μg to 0.051 μg (FIG. 3C). In this embodiment peak 9 was identified as the p24 protein, later used for titer determination, and the resulting profile can be readily used for batch to batch reproducibility and purity assessment. This embodiment also illustrates the excellent reproducibility of the CGE-SDS method, even with various amounts of p24 in the samples proving that the quantitative variation of the p24 protein content did not affect profiling repeatability. In FIG. 3A differing amounts of a p24 standard were spiked into a lentivirus sample with concentration at 1.0×10⁹ TU/mL. FIG. 3B shows excellent linearity of the peak area of the identified p24 peak versus the amount of the p24 standard spiked in. In another aspect, the p24 protein standard was spiked into lentivirus sample at a lower concentration (1×10⁸ TU/mL) (FIG. 3C). The linearity of the peak area vs the spiked in amount of p24 is show in FIG. 3D. With the identification of the p24 peak and its good linearity of the standard addition studies, the p24 is then used for lentivirus titer determination.

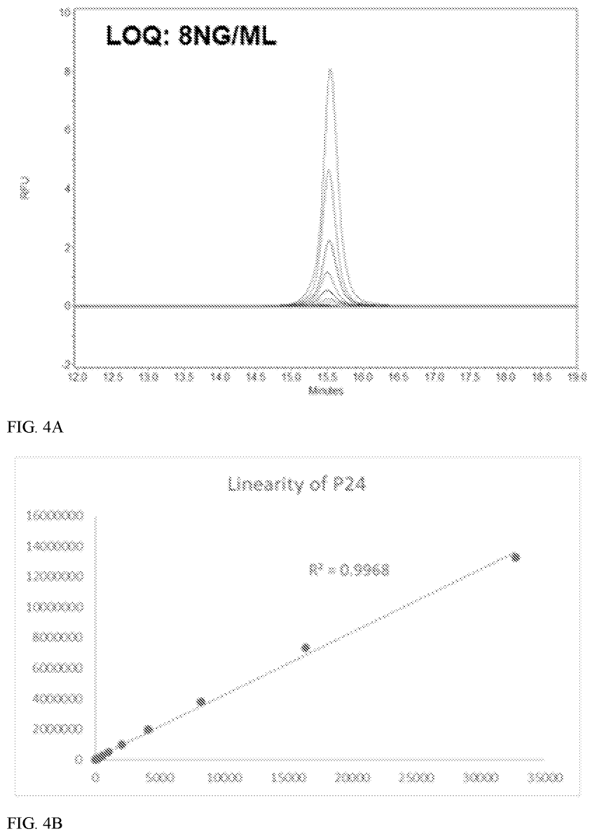

The limit of quantitation (LOQ) values were determined by injecting an increasing amount of standalone p24 standard into the SDS-MW gel-filled capillary column. In various embodiments, the limit of quantification is in a low ng/mL range or is in a sub ng/mL range. As shown in FIGS. 4A and 4B, the limit of quantification is 8 ng/mL.

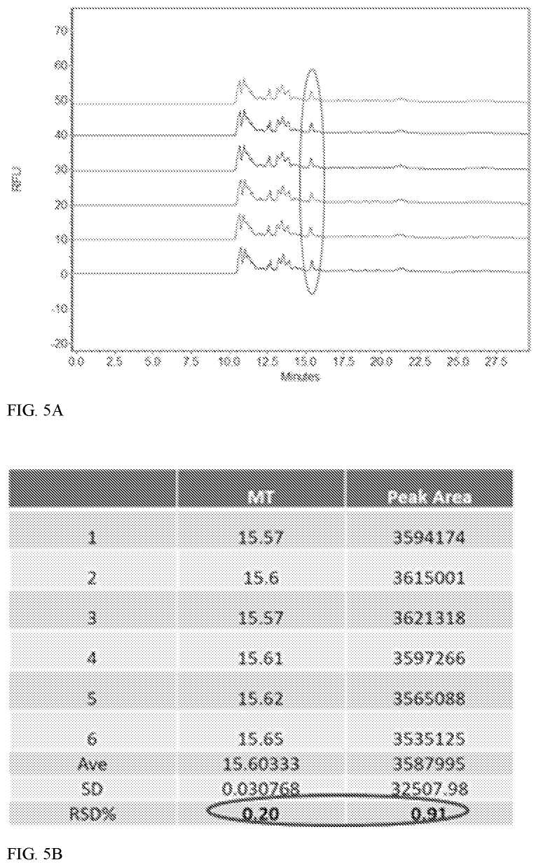

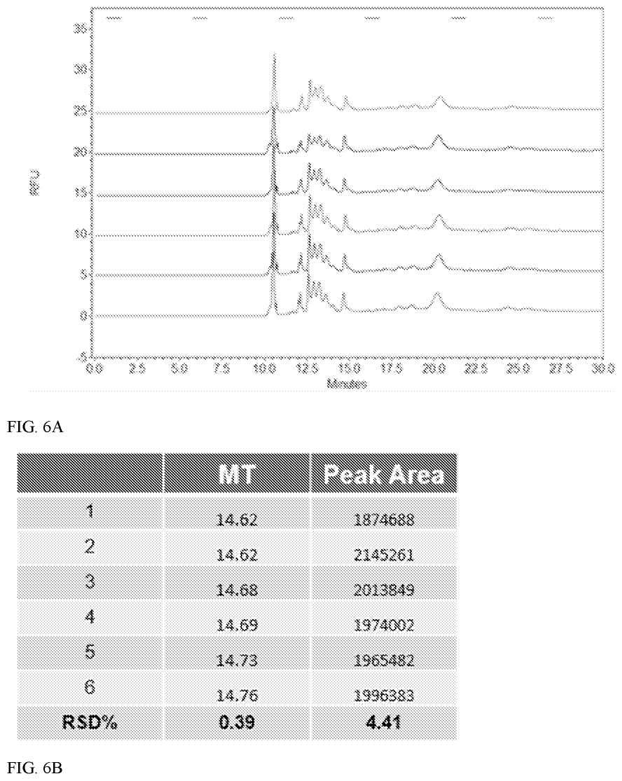

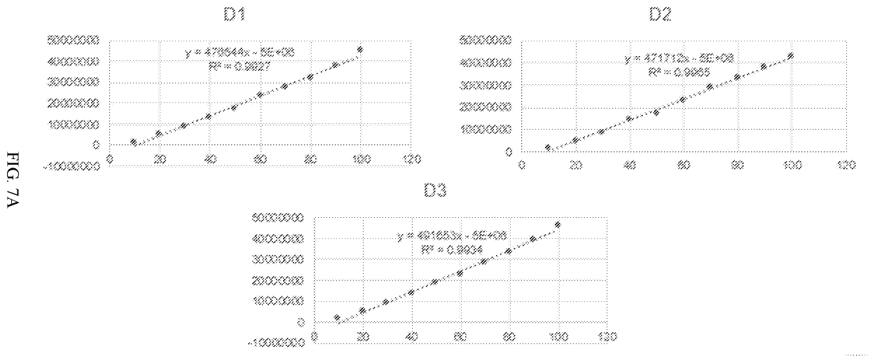

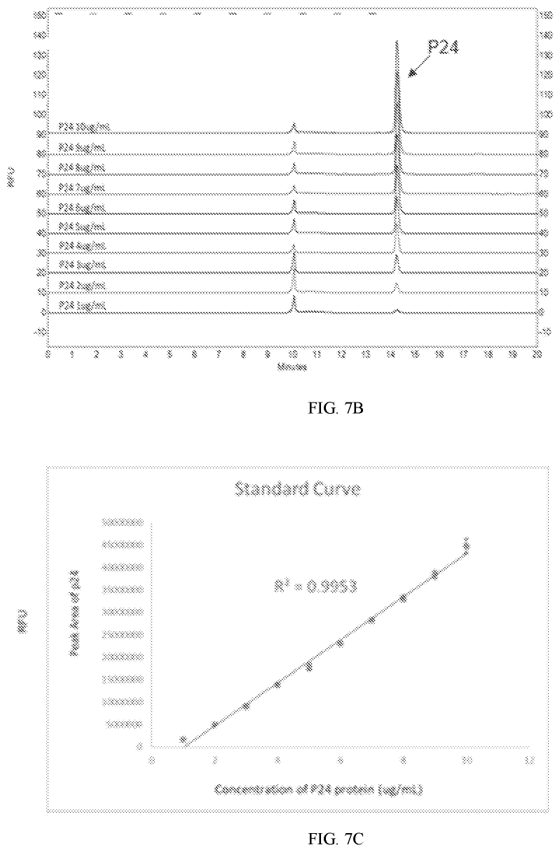

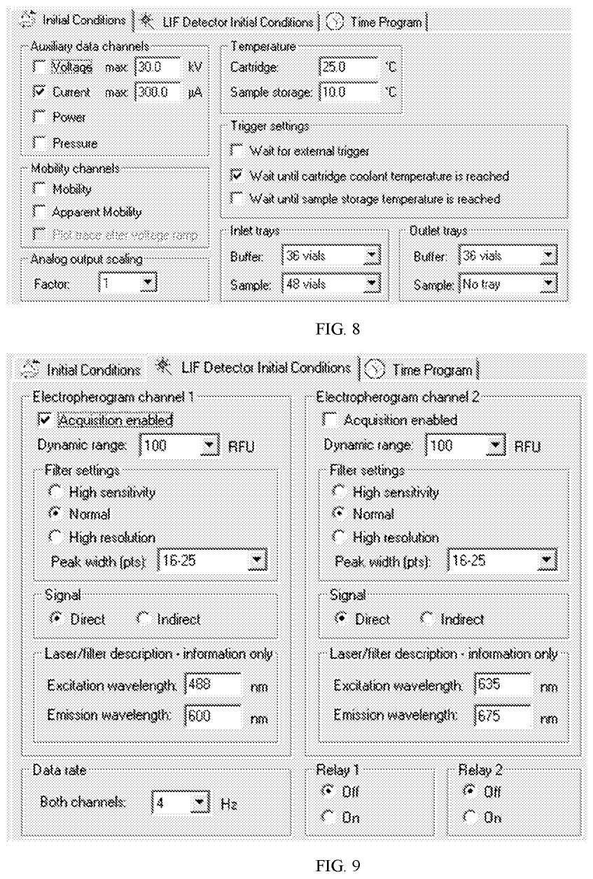

The disclosed methods have several advantages over conventional titer methods including, sensitive titer determination based on precise quantification of the separated protein peak, easy batch to batch reproducibility and purity assessment based on the resulting proteome profile, efficient two-step sample preparation protocol including denaturation and covalent detectable dye labeling of the viral species proteome with no sample cleanup requirement, excellent repeatability with 0.20% RSD on migration time and less than 0.91% on Corrected Peak Area % for six consecutive injections (FIGS. 5A and 5B), excellent repeatability with no more than 1% RSD on migration time and less than 5.00% on Corrected Peak Area % for six sample preparations (FIGS. 6A and 6B), good titer determination detection linearity (r²=0.9953) in the concentration range of 1 μg/mL-10 μg/mL (FIGS. 7A, 7B and 7C). These advantages are present even when multiple different labeled viral protein components are quantified or are quantified on separate days. FIGS. 7A and 7B show the standard curve of a labeled viral protein component quantified on three separate days (FIG. 7A) and the average of these standard curves (FIG. 7C). As illustrated this method still provided for good titer determination detection linearity (r²=0.9953).

Table 1 shows the titer determination of a lentiviral vector sample across 3 days using the average standard curve as shown in FIG. 7C and compares to the titer determined by the ELISA P24 kit.

TABLE 1

| | Titer |
|---|---|
| | (TU/mL) |
| Titer calculated_D1 | 1.45 × 10⁹ |
| Titer calculated_D2 | 1.45 × 10⁹ |
| Titer calculated_D3 | 1.46 × 10⁹ |
| Titer from vendor (ELISA P24 kit) | 1.5 × 10⁹ |

Another embodiment of the disclosure is a kit for quantifying a viral species, wherein the kit comprises: a detectable dye, a CE capillary, wherein the CE capillary is filled with a buffer comprising a polymer matrix, at least one sample buffer, wherein the sample buffer comprises sodium dodecyl sulfate (SDS), at least one internal standard, at least one wash solution, and instructions for use. In an embodiment, the detectable dye is an internal standard. In another embodiment, the kit comprises at least two internal standards, a 10 kDa protein standard and a detectable dye standard. In another embodiment, the kit comprises an acidic wash solution and a basic wash solution. In an embodiment, the instructions for use comprise a method according to at least one embodiment of the disclosure.

### EXAMPLES

### Example 1: Exemplary Sample Preparation and Analysis Method

Materials and Methods

Materials: Pre-made lentivirus (LV-CAG-GFP, PN SL100270) was from SignaGen Laboratories (Rockville, MD), and the HIV1 p24 protein (PN ab127888) was purchased from Abcam (Cambridge, UK). The Chromeo P503 dye (PN 15106) was from Active Motif (Carlsbad, CA). Phosphate Buffered Saline (PBS) 10× bioreagent suitable for cell culture (PN P5493-1L) was from Sigma-Aldrich (St Louis, MO). The SDS-MW Analysis Kit (PN 390953) was from SCIEX (Framingham, MA), including the SDS-MW gel buffer, the acidic and basic wash solutions (0.1 N HCl and 0.1 N NaOH) as well as the SDS-MW sample buffer of 100 mM Tris-HCl (pH 9.0) with 1% SDS.

Sample Preparation

Preparation of the Chromeo P503 working solution: 1 mg of Chromeo P503 dye (lyophilized powder) was reconstituted in 1 mL of methanol. After reconstitution, the dye label can be stored at 2-8° C. for six months according to the manufacturer's instructions. Sample Preparation: 5 μL of lentivirus sample solution (1.5×10⁹ TU/mL) was mixed with 5 μL of sample preparation solution provided in the SDS-MW Analysis Kit and incubated at 70° C. for 10 minutes followed by mixing with 0.5 μL of Chromeo P503 dye working solution and incubated again for another 10 minutes at 70° C. After cooling down to room temperature, 39.5 μL of DI water was added to the reaction mixture, and the diluted sample was transferred to the sample vial for SDS-CGE-LIF analysis.

Capillary Electrophoresis

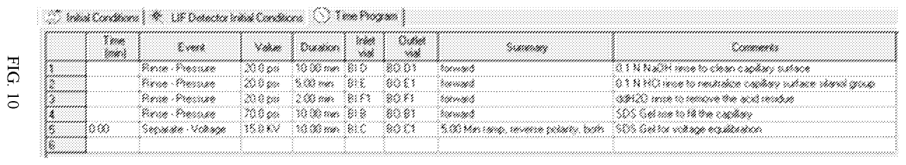

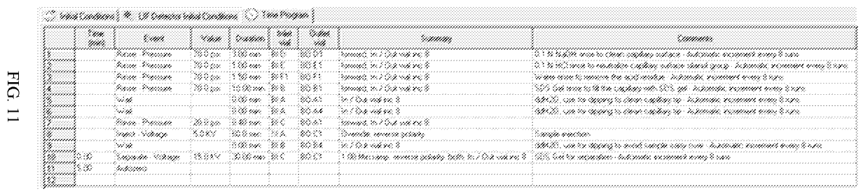

A PA800 Plus Pharmaceutical Analysis CE system (SCIEX) equipped with a laser-induced fluorescence (LIF) detector with a 488 nm solid-state laser and a 600 nm emission filter was used for all separations. The pre-assembled EZ-CE capillary cartridge (SCIEX, PN A55625) had a bare fused-silica capillary with 50 μm I.D. and 30 cm total length (20 cm effective length). The SCIEX universal vials (PN A62251), universal vial caps (PN A62250), and PCR vials (PN 144709) were used for sample loading with the following injection parameters: water stacking at 20.0 psi for 0.40 min and injection at 5 kV for 60 seconds. Data acquisition and analysis were performed using 32 Karat™ software 10. The detailed information of condition, separation, and shutdown methods are shown in FIGS. 9-11.

### Example 2: Titer of a Lentivirus Sample

The titer of the lentivirus sample was determined by using the detection linearity plot in FIG. 7B and found to be 1.45×10⁹ TU/mL, 1.45×10⁹ TU/mL, and 1.46×10⁹ TU/mL as depicted in Table 2 during three consecutive days of analysis.

TABLE 2
Lentivirus titer determined by SDS-CGE and ELISA.

| | Titer |
|---|---|
| | (TU/mL) |
| Titer measured by SDS-CGE_Day1 | 1.45 × 10⁹ |
| Titer measured by SDS-CGE_Day2 | 1.45 × 10⁹ |
| Titer measured by SDS-CGE_Day3 | 1.46 × 10⁹ |
| Titer from vendor (ELISA P24 kit) | 1.5 × 10⁹ |

The titer value measured by ELISA assay was 1.5×10⁹ TU/mL (also shown in Table 1). As a first approximation, we consider this apparent difference in the titer values between the SDS-CGE and ELISA assays of an overestimation of the latter as the ELISA is prone to detect similar epitope proteins in addition to the target protein. This assumption was supported by the complicated profile of the lentivirus proteome (FIG. 2), i.e., the presence of all other proteins in the mixture possibly interfering with the ELISA. The SDS-CGE assay, on the other hand, provided the actual titer value of the sample solely based on quantitating the peak corresponding to the p24 protein (circled peak in FIG. 2), i.e., without any interference of other proteins in the sample. In addition, the resulting profile can be readily used for batch to batch purity assessment.

While the present disclosure has been described with reference to certain embodiments, it will be understood by those skilled in the art that various changes may be made and equivalents may be substituted without departing from the scope of the present disclosure or appended claims. In addition, many modifications may be made to adapt a particular situation or material to the teachings of the present disclosure without departing from its scope. Therefore, it is intended that the present disclosure not be limited to the particular embodiments disclosed, but that the present disclosure will include all aspects falling within the scope of the appended claims.

All patents, patent applications, publications, and descriptions mentioned above are herein incorporated by reference in their entirety.

1. A method for quantifying a viral species, the method comprising the steps of:
preparing at least one labeled viral protein component by incubating a detectable dye with a virial species in the presence of sodium dodecyl sulfate (SDS);
loading the labeled viral protein component onto a capillary electrophoresis (CE) capillary, wherein the CE capillary is filled with a buffer comprising a polymer matrix;
applying a separation voltage to the CE capillary;
detecting at least one labeled viral protein component with a detector, thereby producing a corresponding set of values; and
quantifying a protein of interest in the viral protein component using the corresponding set of values.

2. The method of claim 1, wherein the viral species is a retrovirus.

3. The method of claim 1, wherein the viral species is a lentivirus.

4. The method of claim 3, wherein the viral protein component is a structural protein, non-structural protein, or a residual protein.

5. The method of claim 4, wherein the viral protein component is a structural protein selected from the group consisting of nucleocapsid protein, capsid protein, matrix protein, and envelope protein.

6. The method of claim 5, wherein the structural protein is a p24 capsid protein.

7. The method of claim 1, wherein the viral species is incubated at a temperature between about 40° C. to about 90° C.

8. The method of claim 1, wherein the viral species is incubated for at least 2 minutes.

9. The method of claim 1, wherein the detectable dye is a fluorescent dye.

10. The method of claim 9, wherein the detectable dye has an absorption wavelength and an emission wavelength of between about 480 nm and about 750 nm.

11. The method of claim 9, wherein the detectable dye is a pyrylium-based dye.

12. The method of claim 11, wherein the detectable dye is an amine-reactive pyrylium dye.

13. The method of claim 1, wherein the polymer matrix is selected from the group consisting of crosslinked polymer, linear polymers, slightly branched polymers, linear polyacrylamide, polyethylene oxide, polyethylene glycol, dextran, and pullulan.

14. The method of claim 1, wherein the detector is a fluorescence detector.

15. The method of claim 1, wherein the limit of quantification is in a low ng/mL range.

16. The method of claim 1, wherein the limit of quantification is in a sub ng/mL range.

17. The method of claim 1, wherein the method is used in a high-throughput screening application or a rapid screening workflow.

18. The method of claim 1, wherein at least two labeled viral protein components are prepared.

19. A kit for quantifying a viral species, wherein the kit comprises:
a CE capillary,
a buffer comprising a polymer matrix,
at least one sample buffer, wherein the sample buffer comprises sodium dodecyl sulfate (SDS),
at least one internal standard,
at least one wash solution, and
instructions for use.

20. The kit of claim 19, further comprising a detectable dye.

21. The kit of claim 19, wherein the CE capillary is pre-filled with the buffer comprising a polymer matrix.
